(19) United States
(12) Patent Application Publication
(10) Pub. No.: US 2024/0168021 A1
(43) Pub. Date: May 23, 2024

(54) LATERAL FLOW TEST METHODS

(71) Applicant: Stichting Wageningen Research, Wageningen (NL)

(72) Inventors: Aart VAN AMERONGEN, Veenendaal (NL); Jan Herman WICHERS, Ede (NL)

(21) Appl. No.: 18/549,625

(22) PCT Filed: Mar. 8, 2022
(86) PCT No.: PCT/EP2022/055940

§ 371 Date: Sep. 8, 2023

(30) Foreign Application Priority Data
Mar. 10, 2021 (GB) 2103300.6

(51) Int. Cl. G01N 33/569; G01N 21/64; G01N 33/543

(52) U.S. Cl. CPC G01N 33/56983; G01N 21/6428; G01N 33/54388; G01N 2021/6439; G01N 2333/165; G01N 2469/20

(57) ABSTRACT

A lateral flow test device and method for using same are provided. The device includes epitope test regions having a fragment of an antigenic protein and a full length antigenic protein, and a receptor test region having a receptor for the antigenic protein. Test samples are added to a conjugate pad including free labelled antigenic protein fragments. Neutralising antibodies in the test sample bind to the free labelled antigenic protein, and are detected by the test regions. Binding kinetics and other characteristics of the antibodies can be investigated by monitoring development of label at the test regions, and comparing the same at different test regions. Embodiments describe use for determining antibodies to SARS-COV-2, using RBD fragments, spike proteins, and human ACE2 receptors, at the respective test regions.

### FIELD OF THE INVENTION

The present invention relates to methods and devices for conducting lateral flow tests. In particular, the invention in certain embodiments provides methods and devices for conducting lateral flow tests which may be of assistance in determining the presence and/or titre of neutralising antibodies present in a sample from a patient or subject (which may include non-human subjects).

### BACKGROUND TO THE INVENTION

Lateral flow tests are a rapid and reliable approach to determining the presence of a desired ligand in a given test sample. Typically, a lateral flow device includes a series of regions on a capillary bed which will transport a sample (eg, urine, blood, saliva) containing molecules to be detected between the regions. The capillary bed includes a sample pad, where sample is deposited; a conjugate pad, which includes mobilisable ligand-binding molecules conjugated to a detectable label (“detection” molecules); and a test area, which typically includes immobilised molecules which bind the ligand-binding molecules (“capture” molecules).

Thus, as sample passes from the sample pad to the conjugate pad, the ligand-binding molecules will bind to any ligand present in the sample; these are then detected by being bound in turn at the test area. The detectable label ensures that these can then be detected.

One common use for lateral flow tests is to detect antibodies to a given infection—for example, at present there are many tests in use and in development for antibodies to SARS-COV-2, the virus responsible for the COVID-19 pandemic. Lateral flow tests to detect antibodies typically provide an antigen from the infectious agent conjugated to a detectable label on the conjugate pad (eg, in the case of SARS-COV-2 the spike protein, SP). Antibodies within the sample will bind these labelled antigens. The test area then includes unlabelled antibodies specific for, eg, human IgG antibodies. These will capture the anti-SP antibodies which have bound the antigen.

While rapid and effective, this type of test has a number of disadvantages. One is that the use of anti-human IgG antibodies in the test area means that the test cannot be used for assaying non-human samples, nor can a test using only, say, anti-human IgG antibodies determine the presence of other relevant antibody isotypes (eg, IgA, IgM antibodies which also play a role in the immune response). To do so, the test would need to include multiple different antibodies in the test area.

Further, lateral flow tests of this type may have problems distinguishing between neutralising antibodies—those which bind relevant epitopes so as to reduce or prevent infection—and antibodies which bind different epitopes on the same antigen. For example, the SARS-COV-2 spike protein (SP) includes a receptor binding domain (RBD); antibodies which bind the RBD are more likely to be neutralising antibodies. If the whole SP is used as antigen, a sample may be determined to have a high antibody titre, but no information is given as to the neutralising antibody titre specifically. Conversely, use of the RBD alone can show the existence of potentially neutralising antibodies, but will omit information regarding other anti-SP antibodies which may be present and so indicate previous exposure to SARS-COV-2 or related viruses which has not resulted in anti-RBD antibody production. Further, even when this is addressed in part (for example, by use of both RBD and SP antigens in a test), conventional lateral flow tests are end-point assays in which the kinetic information of the neutralising antibodies is not taken into account. More extensive conventional (non-lateral flow) neutralisation tests are laborious and expensive and require production, purchase and handling of hazardous reagents (e.g. highly infective viruses). In addition, they require sophisticated equipment, high containment laboratories, well trained personnel, and may take days to obtain results. A “gold standard” conventional virus neutralisation test, namely the plaque reduction neutralisation test, requires live cell cultures and live viruses, and measures the ability of the relevant antibody (for example, an antibody present in a sample such as blood, serum, or plasma) to prevent a specific aspect of viral behaviour, for example entry into the cell. Typically the serum or antibody to be tested is mixed with a viral suspension, and incubated to allow the antibody to react with the virus. This is added to a monolayer of host cells. The concentration of plaque forming units can be estimated by the number of plaques (regions of infected cells) formed after a few days. Depending on the virus, the plaque forming units are measured by observation, fluorescent antibodies or specific dyes that react with infected cells.

Certain embodiments and aspects of the present invention are intended to address these and other disadvantages with lateral flow tests.

### SUMMARY OF THE INVENTION

Aspects and embodiments of the present invention are based around the use of a “double antigen” lateral flow test—that is, the same antigen is present (labelled) on the conjugate pad, and (unlabelled) on the test area. This antigen is used to detect the presence of a relevant antibody in a test sample, taking advantage of the fact that a single Y-shaped antibody includes two identical antigen binding domains. This has the advantage that no species-specific anti-Ig antibodies are required for the test, so that it is not restricted to, say, human samples; and that the same test can detect presence not only of IgG antibody isotypes, but of other antibody isotypes such as IgM or IgA as well.

Aspects and embodiments of the invention are further based around the use of an “antigen-receptor” lateral flow test, in which an additional test area includes a receptor molecule which interacts with the antigen. When neutralising antibodies are present in the test sample, these will prevent the antigen from interacting with the receptor and so no or reduced signal will be obtained, compared with the situation when neutralising antibodies are not present in the test sample, and the antigen is able to interact with the receptor. Comparison of test results from the antigen test area and the receptor test area may be useful in quantitative measurement of results.

In further aspects and embodiments of the invention, the lateral flow test further comprises a kinetic element—that is, the kinetics of antibody-antigen binding are determined over time, rather than just a single end-point analysis; for example, by monitoring the time over which a result in the test area is obtained.

Thus, according to an aspect of the present invention, there is provided a method for detecting the presence in a test sample of a neutralising antibody to a neutralisation epitope of an antigenic protein, the method comprising:
applying a test sample to a sample receiving region of a lateral flow test device, the device comprising a solid support structure including the sample receiving region, a conjugate pad, an epitope test region, and a receptor test region; wherein the conjugate pad comprises mobilisable neutralisation epitope conjugated to a detectable label, the epitope test region comprises immobilised neutralisation epitope which is not conjugated to a detectable label, and the receptor test region comprises immobilised receptor to the antigenic protein; and wherein the solid support structure is configured to permit liquid to flow from the sample receiving region via the conjugate pad to the test regions;
allowing the test sample to migrate to the conjugate pad and contact the neutralisation epitope to form labelled antibody-epitope complex if antibody is present;
   allowing the test sample including labelled antibody-epitope complex or labelled epitope to migrate to the epitope and receptor test regions and contact the unlabelled antigen epitope or receptor respectively to form a complex, the label thereby providing a detectable signal; and
detecting a signal at the epitope test region, wherein presence of a signal is indicative of the presence of a neutralising antibody to said neutralisation epitope of an antigenic protein.

In preferred embodiments, the epitope and receptor test regions are arranged such that the sample may contact either or both regions independently—for example, the two regions may be arranged as spots on a substrate adjacent the conjugate pad. It is preferred that the sample does not contact the test regions sequentially, as to do so would risk depleting some components from the sample, thereby potentially interfering with a subsequent test. If sequential testing is however being used, the preferred order is first the receptor test region and then the epitope test region. If, instead of lines, spots are used, the various spots can be placed next to each other (or zig-zag over two rows), thereby not influencing each others interaction with the mobilisable neutralisation epitope. Each immobilised receptor or epitope will have the chance to interact with the full range of sample (eg blood/serum/plasma) components, which is not the case if lines (receptors/epitopes) are placed sequentially, thereby changing the concentration/ratio of these components if particular components will be bound at the first line, the second line, etc. Also the kinetic parameters for each spot will be related to the full sample and not to an essentially changed composition of components at line 2, 3 and higher.

The method may further comprise the step of detecting a signal at the receptor test region; and optionally comparing said signal to the signal detected at the epitope test region. In principle, presence of a signal at the receptor test region is indicative of the absence of a neutralising antibody to said neutralisation epitope of an antigenic protein in the test sample, as the epitope is free to bind to the receptor. In practice however, there is likely to be signal at both test regions when a neutralising antibody is present, as at least some free labelled epitope may remain unbound by the antibody and available to bind the receptor. Comparison of the signals or signal kinetics at each test region may therefore be useful in quantifying the titre of the antibody if present. Conversely, if no specific antibody is present, signal at the epitope test region should be absent, while a strong signal will be detected at the receptor test region.

Where reference is made to a part of the device which comprises “mobilisable” or “immobilised” molecule (eg, the neutralisation epitope), any suitable means may be used to incorporate said molecule on said part of the device, and the skilled person will be aware of suitable techniques and protocols. It will be understood that a “mobilisable” epitope is one which will normally be retained on the conjugate pad prior to use of the device, but which will be carried by lateral flow of the sample away from the conjugate pad to the test region in use. An “immobilised” epitope is one which will normally be retained in the test region in use, and will hence capture relevant antibodies contained within the flowing sample.

The test sample may comprise a body fluid (for example, blood, serum, saliva, mucus); may be a liquid sample derived from a swab or other test (for example, cellular and non-cellular material suspended in a liquid); or may be a liquid sample derived from a solid sample (for example, a solution of the solid sample or some component thereof).

The step of detecting the signal may comprise quantitatively detecting the signal (for example, by measuring intensity and/or number of signals on a detector), and in preferred embodiments, quantitatively detecting development of the signal over time. For example, the intensity of the label may be measured at a series of time points, or continuously, and the change in intensity over time determined. Such measurements may allow the kinetics of antibody binding to be measured or determined—for example, at its crudest the time taken to reach a particular intensity threshold may be considered a measure of binding kinetics; or the rate of change of intensity of the signal. In certain embodiments, the kinetic information (as represented by, for example, the change in intensity of the signal over time) is preferably correlated with the neutralising titre of the test sample in a neutralisation test, for example in a virus neutralisation test.

The neutralisation epitope of an antigenic peptide may be embodied by a peptide sequence. In preferred embodiments, the neutralisation epitope consists of a fragment of a full length protein, said fragment being one which is known to be recognised by neutralising antibodies. In such embodiments, the conjugate pad may further comprise mobilisable full length protein conjugated to a detectable label; and the epitope test region may further comprise immobilised full length protein not conjugated to a detectable label. This arrangement allows potentially neutralising antibodies (which bind to the fragment) to be compared with the total titre of antibodies which bind to the protein—in preferred embodiments, the kinetics of both binding reactions are determined and compared. In some embodiments, however, a combination of the epitope and full length protein are not present in the conjugate pad, but are both present in the test region(s). The signal from the test region of antibodies binding the full length protein can be compared with antibodies binding the epitope, while the conjugate pad may comprise either the epitope alone or the full length protein alone. It is preferred however that the conjugate pad comprises full length protein alone.

Likewise, where both epitope and receptor test regions are present, kinetics of each binding reaction (antibody to fragment and full length protein, and/or fragment and full length protein to receptor) may be determined and compared.

The epitope test region may comprise first and second separate epitope test regions, each of which includes either the fragment or the full length protein. In some embodiments first and second detectable labels are used such that the fragment and the full length protein may be distinguished; although in preferred embodiments only a labelled full length protein can be used, since antibodies to both the fragment and the full length protein will bind the labelled protein. The full length protein may be a SARS-CoV-2 spike protein (SP), and the fragment the receptor binding domain (RBD) of the spike protein.

The receptor for the antigenic protein may be any receptor to which the protein would normally bind in vivo. In preferred embodiments, the antigenic protein is from a pathogen, and the receptor is a host receptor for the pathogen protein. For example, where the antigenic protein is SARS-COV-2 spike protein, the host receptor may be human ACE2 receptor.

The detectable label may be a dye particle, a carbon particle, a fluorescent label, a latex particle, a gold particle, a magnetic particle, or the like.

Where the epitope test region(s) comprise(s) both the fragment and the full length protein, then the binding kinetics of both fragment and full length protein may be compared. For example, the ratio of the final intensity of signal from each may be determined; and/or the time taken to develop a fraction of the final signal intensity—eg, 50%—of each may be determined and compared. In another example, the initial slope of the intensity curve at the or each test region during binding may be used as a measurement to determine antibody affinity and/or titre.

In embodiments of the invention in which multiple test regions are provided (either multiple epitope test regions, or one or more epitope test regions and one or more receptor test regions), these may be sequential test regions, for instance sequential test lines (in which the sample will pass over a first test line, then a second test line, etc, in sequence); or preferably may be parallel or otherwise non-sequential test regions, for instance multiple test spots (in which the sample will pass over the multiple spots either simultaneously or in different sequential flows). The use of non-sequential test regions avoids the possibility that the sample may be depleted of antibodies which bind the full length protein after passing over the epitope. In some embodiments where multiple epitope test regions are provided, these may include variants of a viral protein; for example, different genetic lineages and variants of the SARS-COV-2 virus may be included on a single test. This can be useful in determining whether a given variant affects neutralising capability of an antibody so as to monitor, for example, vaccine efficacy. Where multiple epitope test regions are present which include epitopes from more than one antigenic protein—for example, from different viruses—then corresponding receptor test regions may be provided if the antigenic proteins have different receptors.

In some embodiments, the neutralisation epitope is a host-interacting domain of a pathogen protein. The pathogen protein may be a host-interacting protein or a toxin. The pathogen protein may be a viral protein, bacterial protein, phage protein, fungal protein; may be a parasite protein or an allergen. In particular embodiments, the pathogen protein is the SARS-COV-2 spike protein (SP), and the neutralisation epitope is the receptor binding domain (RBD).

The solid support may further comprise a control region. The control region may comprise a control ligand-binding molecule; for example, a molecule which binds the labelled neutralisation epitope from the conjugate pad (preferably not the antibody which is to be detected; and preferably also not a receptor to the antigenic protein). An alternative control molecule can also be constructed by using a detectable label conjugated to a “nonsense molecule”; that is, a molecule which is not related to the antigenic protein. The control line/spot can use a binding ligand specific to that nonsense molecule; this provides the advantage of a constant signal intensity, because the amount of detection label captured is not influenced by the antigen-antibody interaction of interest in the very same assay.

In further aspects of the invention, the above methods may be used for measuring severity of a pathogenic infection in a patient, by detecting a signal at the epitope test region wherein the neutralisation epitope is derived from a pathogen protein. In other aspects, the methods may be used for determining whether a subject has been exposed to a pathogenic infection or for detecting immunity to a pathogenic infection in a patient In a preferred embodiment, the pathogen is SARS-COV-2.

In particular embodiments, the methods may be used for differential identification of immune responses to infections in a patient; wherein the epitope test region carries multiple antigenic epitopes derived from multiple different pathogens. For example, the multiple different pathogens may include one or more of SARS-COV-2, MERS, SARS-CoV, Influenza Virus, Respiratory Syncytial Virus; this may be useful in distinguishing between different respiratory infections.

Other embodiments permit the methods to be used for determining efficacy of a vaccine, wherein the sample is obtained from a subject that has been administered a vaccine against a pathogen from which the neutralisation epitope is derived; and detection of a signal at the epitope test region is indicative of an efficacious vaccination in a subject. In a preferred embodiment, the pathogen is SARS-COV-2; in some embodiments, multiple variants of a neutralisation epitope may be included, so as to permit determination of neutralising antibody efficacy against such variants.

In any such methods, detecting the signal may involve detecting the presence of a signal or quantitatively detecting development of the signal, e.g. over time, or by comparing the binding kinetics of both fragment and full length protein, or by comparing the ratio with a threshold ratio, as described herein.

Also provided herein is a lateral flow test device comprising a solid support structure including a sample receiving region, a conjugate pad, an epitope test region, and a receptor test region; wherein the conjugate pad comprises a mobilisable neutralisation epitope of an antigenic peptide conjugated to a detectable label, the epitope test region comprises immobilised neutralisation epitope which is not conjugated to a detectable label, and the receptor test region comprises immobilised receptor to the antigenic protein; and wherein the solid support structure is configured to permit liquid to flow from the sample receiving region via the conjugate pad to the test regions. Liquid may flow to multiple test regions simultaneously from the conjugate pad.

The neutralisation epitope of an antigenic peptide may be embodied by a peptide sequence. In preferred embodiments, the neutralisation epitope consists of a fragment of a full length protein, said fragment being one which is known to be recognised by neutralising antibodies. In such embodiments, the conjugate pad may further comprise the mobilisable full length protein conjugated to a detectable label; and the epitope test region may further comprise the immobilised full length protein not conjugated to a detectable label. The epitope test region may comprise both the fragment and the full length protein in a single epitope test region, or may comprise first and second separate test regions, each of which includes either the fragment or the full length protein. Where both the fragment and the full length protein are in a single test region, then in some embodiments first and second detectable labels are used such that these may be distinguished; although in others only a labelled full length protein can be used, since antibodies to both the fragment and the full length protein will bind the labelled protein. Where separate test regions are provided, the labels may be the same or different.

The lateral flow test device may further comprise, or may be provided in combination with, a reader device which is capable of detecting the detectable label.

Other aspects of the invention include an apparatus for detecting the presence of neutralising antibodies to a pathogen protein (preferably SARS-COV-2) in a sample comprising a lateral flow assay device, wherein the lateral flow assay device comprises:
a region for receiving a sample;
a membrane, wherein the membrane comprises
a conjugate pad, wherein deposited on the conjugate pad is a plurality of mobilisable first detection antigens comprising a neutralisation epitope derived from the pathogen protein (preferably SARS-COV-2 spike protein (SP)), wherein the first detection antigen is conjugated to a labelled particle;
at least one epitope test region, wherein immobilised at the at least one epitope test region is a plurality of first capture antigens, wherein the first capture antigens comprise said neutralisation epitope which is not conjugated to a labelled particle; and
a receptor test region, wherein immobilised at the receptor test region is a plurality of receptors which in vivo interact with the pathogen protein, which are not conjugated to labelled particles.

The lateral flow assay device in some embodiments further comprises a second labelled detection antigen (preferably a full length SARS-COV-2 peptide, more preferably a full length SARS-COV-2 spike peptide), and a second capture antigen, comprising an unlabelled second detection antigen. In other embodiments, the device may comprise only a first labelled detection antigen, but further comprises a second capture antigen, said second capture antigen preferably consisting of a full length pathogen protein.

The receptors are preferably host receptor proteins, and may be human ACE2 receptors.

The apparatus may further comprise a spectral sensor suitable for detecting the label of the labelled detection antigen.

Further provided herein is a method for detecting the presence of neutralising antibodies to a pathogenic protein using the apparatus as described herein, the method comprising
applying a sample to the receiving region;
allowing the sample to migrate to the conjugate pad and contact the detection antigen(s) to form labelled antibody-detection antigen complex(es);
   allowing the antibody-detection antigen complex and/or uncomplexed labelled detection antigen to migrate to the at least one epitope and receptor test regions and contact the capture antigen(s) or receptor respectively where a labelled detection antigen-antibody-capture antigen complex is immobilised at the epitope test region and a detectable signal is formed; and
detecting a signal at the first epitope test region, wherein presence of a signal is indicative of the presence of neutralising antibodies.

The method may further comprise detecting a signal at the receptor test region; and optionally comparing signals from the epitope and receptor test regions.

In preferred embodiments, the apparatus comprises first and second detection antigens and first and second epitope test regions as described, and the method comprises allowing the sample to migrate to the conjugate pad and contact the first and second detection antigen(s) to form a first labelled antibody-detection antigen complex and a second labelled antibody-detection antigen complex;
allowing the sample to migrate to the first epitope test region and contact the first capture antigen where a first labelled detection antigen-antibody-capture antigen complex is immobilised and a first detectable signal is formed;
allowing the sample to migrate to the second epitope test region and contact the second capture antigen where a second labelled detection antigen-antibody-capture antigen complex is immobilised and a second detectable signal is formed; and
detecting a first and second signal, where the ratio between the first and second signal provides a ratio of antibody binding to the first antigen: a second antigen.

In some embodiments, the method can further comprise comparing the ratio with a threshold ratio, wherein a ratio that is higher than the threshold ratio indicates a protective level of neutralising antibodies to the pathogen in the sample.

Also provided is a method for detecting immunity to a pathogen infection in a patient using the apparatus described herein, the method comprising
applying a sample to the receiving region;
allowing the sample to migrate to the conjugate pad and contact the detection antigen(s) to form labelled antibody-detection antigen complex(es);
allowing the antibody-detection antigen complex to migrate to the at least one epitope test region and contact the capture antigen(s) where a labelled detection antigen-antibody-capture antigen complex is immobilised and a detectable signal is formed;
allowing antibody-detection antigen complex and/or uncomplexed labelled detection antigen to migrate to the receptor test region; and
detecting a signal at the first epitope test region, wherein presence of a signal at the first test region is indicative of immunity to SARS-COV-2.

Also provided is a method for evaluating the efficacy of a vaccine using the apparatus described herein, the method comprising
applying a sample to the receiving region, wherein the sample is obtained from a subject that has been administered the vaccine;
allowing the sample to migrate to the conjugate pad and contact the detection antigen(s) to form labelled antibody-detection antigen complex(es);
allowing the antibody-detection antigen complex to migrate to the at least one epitope test region and contact the capture antigen(s) where a labelled detection antigen-antibody-capture antigen complex is immobilised and a detectable signal is formed;
allowing antibody-detection antigen complex and/or uncomplexed labelled detection antigen to migrate to the receptor test region and
detecting a signal at the first epitope test region, wherein presence of a signal at the first test region is indicative of an efficacious vaccination in a subject.

Migration to the epitope test region and migration to the receptor test region can take place sequentially or (preferably) simultaneously.

Other aspects of the invention include the following embodiments:
a. A method for detecting the presence in a test sample of a neutralising antibody to a neutralisation epitope of an antigenic protein, the method comprising:
   applying a test sample to a sample receiving region of a lateral flow test device, the device comprising a solid support structure including the sample receiving region, a conjugate pad, and a test region; wherein the conjugate pad comprises mobilisable neutralisation epitope conjugated to a detectable label, and the test region comprises immobilised neutralisation epitope which is not conjugated to a detectable label; and wherein the solid support structure is configured to permit liquid to flow from the sample receiving region via the conjugate pad to the test region;
   allowing the test sample to migrate to the conjugate pad and contact the neutralisation epitope to form labelled antibody-epitope complex;
   allowing the labelled antibody-epitope complex to migrate to the test region and contact the unlabelled antigen epitope to form an antibody-labelled epitope-unlabelled epitope complex, the label thereby providing a detectable signal; and
   detecting a signal at the test region, wherein presence of a signal is indicative of the presence of a neutralising antibody to said neutralisation epitope of an antigenic protein.
b. The method of embodiment a), wherein the antigenic protein is a pathogen protein, and when used:
   a) for measuring severity of a pathogenic infection in a patient;
   b) for determining whether a subject has been exposed to a pathogenic infection;
   c) for differential identification of immune responses to infections in a patient; wherein the test region comprises immobilised neutralisation epitopes from a plurality of different pathogens or pathogen variants;
   d) for determining efficacy of a vaccine, wherein the sample is obtained from a subject that has been administered a vaccine against the pathogen; and detection of a signal at the test region is indicative of an efficacious vaccination in a subject.
c. A lateral flow test device comprising a solid support structure including a sample receiving region, a conjugate pad, and a test region; wherein the conjugate pad comprises mobilisable neutralisation epitope conjugated to a detectable label, and the test region comprises immobilised neutralisation epitope which is not conjugated to a detectable label; and wherein the solid support structure is configured to permit liquid to flow from the sample receiving region via the conjugate pad to the test region.

### BRIEF DESCRIPTION OF THE DRAWINGS

These and other aspects of the invention will now be described, by way of example only, and with reference to the accompanying drawings, in which:

FIG. 1 shows two alternative (partial) first arrangements of a lateral flow test

FIG. 2 shows an arrangement of two test lines in which the same label is used on both the antigen fragment, and the full length antigen

FIG. 3 shows an arrangement in which two different label colours are used

FIG. 4 shows an arrangement of three test lines in which the same label is used on the host receptor protein, the antigen fragment and the full length antigen

FIG. 5 shows an arrangement using non-overlapping test spots rather than test lines

FIG. 6 shows a further arrangement using test spots to detect variants of a pathogenic virus

FIG. 7 shows a further arrangement using test spots to detect variants of a pathogenic virus and including multiple identical receptor test spots

FIG. 8 shows a further arrangement using multiple different test spots and multiple different receptor test spots

### DETAILED DESCRIPTION OF THE INVENTION

A method is proposed to rapidly assess and quantify the neutralising potential (titer) of specific antibodies in samples such as blood, serum, plasma, and other fluids containing antibodies (saliva, sputum, mucus) by running and analyzing a lateral flow assay in a kinetic mode and by applying a double antigen approach. This “double antigen approach” means that the protein involved (or a relevant fragment thereof) is used as both detection and capture ligand. Such a relevant fragment of the protein may be a receptor binding region (e.g. SARS-COV-2 Receptor Binding Domain from the Spike Protein), a toxicity-inducing region, a cancer-related activity region, or any other protein region the activity of which can be neutralised by antibodies. By the real-time recording of signals resulting from the binding of antibodies to the detection and capture proteins/protein fragments, kinetic information is obtained that correlates with the neutralising potential of the antibodies involved, i.e., a combination of the number of antibody interactions and the strength of the individual interactive forces. Signals can be recorded by a suitable lateral flow test reader.

The double antigen approach takes advantage of the fact that antibody molecules include multiple identical antigen binding domains, permitting a single antibody molecule to bind to two antigen molecules, or to different surface positions on the antigen if it has repetitive structures such as bacteria and viruses. Conventionally the set up of immunoassays (including lateral flow assays) to detect specific human antibodies against a pathogen (bacterial cell, viral particle) makes use of different detection and capture ligands—for example, a pathogen specific protein (detection ligand), such as SP in the case of SARS-COV-2, and anti-human IgM, IgG and/or IgA antibodies as capture ligand(s). This means that for detection of the various antibody classes (IgM, IgG, IgA) three different capture ligands/regions will be necessary. In commercial assays IgM and IgG are often measured. However, IgA is an important immunoglobulin class that operates at the body's inside/outside border. Especially in the case of SARS-COV-2 that invades the body in the lungs' alveoli, a specific IgA response is an important defence mechanism. The advantages of the double antigen approach are twofold: 1) it is suitable to detect all antibody classes, including IgA, and 2) it is species-independent; there is no need to use anti-human (or other mammal) antibodies specific for IgM, IgG, or IgA. This approach should therefore improve the detection capabilities of such assays, and may lead to greater accuracy of persons positively identified as having had COVID-19. In addition, the same test can be used to detect specific antibodies in animals infected by the same pathogen. The test principle can, therefore, be used in a OneHealth approach and be instrumental in elucidating zoonotic routes of pathogens.

A person that has been infected with SARS-COV-2 will have antibodies specific for SP. The serum antibody titer of this person (i.e., the inverse of the serum dilution that results in 50% signal reduction in an ELISA) can be high, which means that the serum can be diluted many times and still give a signal. This would give the impression that this person is well protected. However, a more specific question would be whether this serum contains a sufficient number of neutralizing antibodies that as well as binding to SP also bind SP in such a way that the virus will be prevented from binding to the human receptor ACE2. This means that these antibodies must bind to the Receptor Binding Domain (RBD) of SP, or at least in the vicinity of this part on the SP surface to be of steric hindrance in the interaction between SP and ACE2. Therefore, a test that focuses on the detection of neutralizing antibodies with respect to the overall SP response adds valuable information 1) with respect to protection of a person to a second attack by the virus, or 2) for the assessment whether a vaccine induces neutralizing antibodies in a sufficiently high titer.

Conventional lateral flow tests measure binding at a single time point, once the reaction has concluded. However, determining the rate of binding or other kinetic data by recording a signal at multiple time points (or continuously) can provide further valuable information on antibody response. The combination of number and individual binding strengths (affinity) of RBD-binding antibodies and the ratio of these antibodies to all antibodies that bind SP may be important to the level that people are immune to a second (and further) attack by the virus. (And similar reasoning may apply to other neutralising antibodies to other pathogens or disorders, where it could be valuable to distinguish between neutralising antibodies and other antibodies which merely bind the same target protein but do not prevent illness).

There are existing alternatives for measurement of neutralising antibodies, but these suffer from a number of disadvantages. The gold standard is the plaque reduction neutralisation test, which requires live cell culture and live virus, as well as taking several days to provide a result. Other alternatives are typically ELISA-based and, therefore, end-point assays. This means that the interaction between (neutralising) antibody and SP and the further (reduced) interaction between SP and ACE2 is judged after a particular time period, often 30 to 60 minutes initial incubation followed by second antibody incubation and colouring step. It also implies that affinities of antibodies (kₒₙ and k_{off}; affinity constant) is of less importance, since there is ample time for the antibodies to bind to SP; also antibodies with a slow on-rate or with a lower binding strength may interact in the assay. However, in real life antibodies should react to pathogens intruding the body within seconds; there is no ‘incubation time’ provided. Therefore, diagnostic methods that allow for immediate/short term interaction of antibody molecules with their target much more resemble characteristics necessary to combat infections by pathogens such as viruses, bacteria, parasites and toxins.

Diagnostic methods that are flow-based fulfil these requirements, in that a sample flows over a binding region. Well-known examples are methods based on optical principles such as Surface Plasmon Resonance and interferometry. These sophisticated methods also enable the assessment of kinetic data (kₒₙ and k_{off}; affinity constant) and, therefore, provide a much better appraisal of antigen and/or antibody characteristics in view of physiological functionalities.

With respect to flow and the short period of interaction possibilities between reactants the lateral flow technology in nitrocellulose membranes resembles these sophisticated optical methods, although the traditional lateral flow assay is typically an end-point assay; the coloured line is read after a fixed timepoint (10, 20, max 30 minutes).

Lateral flow microarray technology has been developed in which small spots are printed on the nitrocellulose membrane instead of lines sprayed. Compared to line assays with a maximum of 4 to 5 lines the microarray lateral flow test can accommodate up to 25 spots, i.e., different tests, while being identical in assay execution and performance. To enable interpretation of results, a real-time video reader can be used to record the colour development of specific spots on nitrocellulose membranes, thereby generating kinetic data on the interaction between reactants in the test, e.g. antibodies from a sample and antigens on a coloured conjugate and on the test membrane, or any other combination of antigen and antibodies. The slope of the intensity curve as recorded over time reflects 1) the number of interactions between antigen and antibody molecules and 2) the combined affinities of all antigen-antibody interactions. It, thus, adds relevant information on the interaction between these reactants with respect to the physiological situation.

The colour intensity further adds information on the total number and overall strength of the interactions.

EP 3 279 662 may be of relevance in permitting determination of the correlation between lateral flow kinetic data and neutralisation titers, and the reader is referred to that publication for further information.

It is possible to combine multiple antigens in a single test by use of an array of capture antigens and multiple detection antigens. Note that the detection antigens need not be in an array, since the same sample can be applied to a conjugate pad comprising multiple detection antigens. In some embodiments, even the capture antigens need not be in an array and could be in the same location, provided the labels used on each antigen are distinguishable. For example, both SP and NP are important antigens with respect to the question whether a person has developed an anti-SARS-COV-2 immune response. In addition, other antigens can be added that would give valuable information on infections by other viruses: for example, SARS-COV(1), MERS, influenza virus, etc. Such a test could rule out the possibility that a person has been infected by SARS-COV-2 and instead suffers from another, less dangerous virus. It is anticipated that such tests will be extremely valuable in the event of future waves of SARS-COV-2 infections, or if the virus becomes endemic in future.

The majority of lateral flow tests use a “line assay”—that is, the result is indicated by a label becoming visible as a line extending across the width of the test strip. It is possible to record kinetic data with a line assay using appropriate readers. However, since a line covers the width of the nitrocellulose membrane completely, use of multiple line assays in a single test is difficult, since the sample has to flow across the first, second, third, etc, test lines in sequence. As such the concentration of the conjugate that is left to specifically interact on the second, third and further lines cannot be standardized, as it will have been depleted by contact with preceding lines. Thus, kinetic data of the first line can be compared to other tests, but this will not be the case for the second and further lines. An option is to take the kinetic data of the first line and the intensity ratio of the first and second line as input information to acquire an algorithm that links this information to the neutralizing activity of the serum. If, however, an array of spots is being used, then the spots can be arranged so that the sample flow path does not overlap. Hence all spots can be used for kinetic data acquisition. Then, it is possible to differentiate at least 8 spots, i.e., 8 different antigens.

The new neutralisation test is based on the interaction of specific antibodies with (a fragment of) the protein of interest that is relevant for its functionality/activity. No other ligands (e.g., anti-species antibodies) are involved. The test is rapid, quantitative and real-time by kinetically measuring the signal that results from the interaction between antibody molecules and the protein (fragment) of interest in a double antigen format.

While simple detection of binding, eg, by determining signal intensity, permits a crude assessment of the presence of neutralising antibodies, comparison between an antigen fragment and a full length antigen provides improved results. The sensitivity is expected to be improved still further as kinetic measurements are incorporated into the assessment. We believe that further developments will permit accurate neutralisation titers to be calculated by determining a correlation curve based on results from conventional neutralisation tests and measurements with the present invention.

The invention thus provides a number of important advantages:

Application of the double-antigen approach, i.e., the antigen both as detection and capture ligand, enables the detection of all antibody classes at the same time and on the same line or spot and renders a test species-independent; without any adaptation also animals can be tested to assess whether they are hosting the pathogen.

By using a combination of antigen fragments and full length antigens (eg, RBD and SP for SARS-COV-2) in a lateral flow assay the intensity ratio of signals from each can be used to provide information of the neutralizing capacity of an antiserum. This information is valuable to individuals and medical professionals and also to pharmaceutical companies to assess whether people vaccinated develop neutralizing antibodies.

Acquisition of kinetic data on the intensity increase of the RBD line in a lateral flow test could, in combination with the intensity ratio of the signals of the RBD and SP lines, give information that may be correlated to neutralization titers of an antiserum.

Acquisition of kinetic data of all spots in an array on a lateral flow membrane will yield information on each of the antigens printed in the array. This information can be used as input to determine an algorithm that links this information to the neutralizing activity of the serum (neutralization titer). Since the spots can be arranged so that the sample does not have to contact them in sequence, the intensity and kinetic data of the SP (and other) spot(s) will not be influenced by the RBD spot. Since lines span the total width of the nitrocellulose membrane antibodies binding to the RBD line will be depleted before the sample reaches the second, SP line, thereby influencing the second signal. However, spots can be positioned in such as way that there is no influence of other spots.

The above-mentioned test possibilities are not restricted to SARS-COV-2, but can be applied to any infectious disease (parasites, bacteria, viruses), as well as to bacterial toxins and cancers. We further believe that kinetic information could be helpful in allergy testing as well (In allergy IgE and IgG4 antibodies are involved). Present diagnostics in the assessment of allergy are far from ideal. If sufficiently sensitive lateral flow tests can be developed, this information is likely to be correlated to a higher extent to clinical symptoms than end-point immunoassays.

Representative schematics of certain potential tests are shown in the Figures.

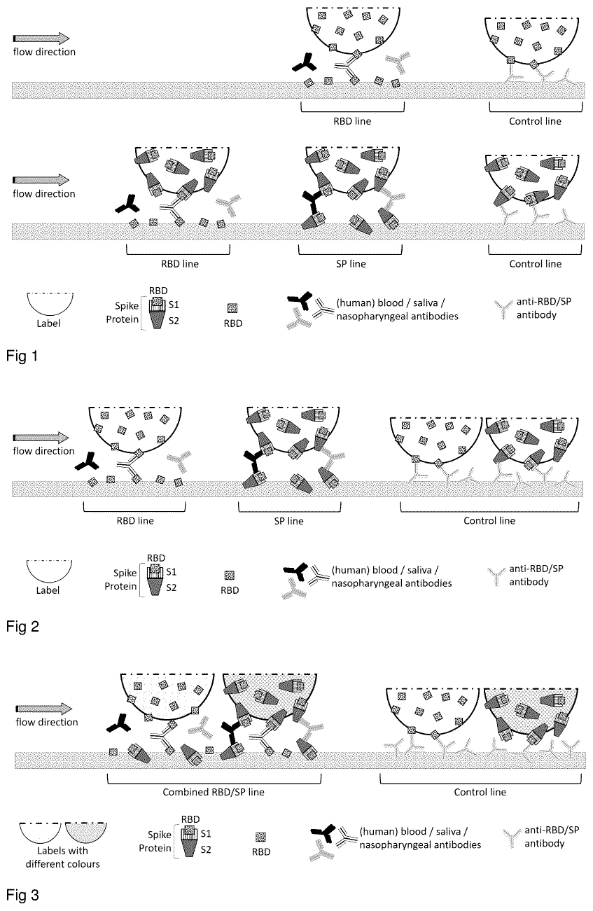

FIG. 1 shows two alternative (partial) first arrangements, in which an RBD label and one RBD test line (upper panel), or an SP label and two test lines, RBD and SP respectively (lower panel), both including a control line are used. Note that these schematics are only partial, in that certain features—such as the conjugate pad—are not shown here. However, the skilled person will be aware of the general construction of lateral flow tests.

FIG. 2 shows an arrangement of two test lines, the RBD antigen fragment and the SP full length antigen respectively, and a control line. In this arrangement an RBD and an SP label are used.

FIG. 3 shows an arrangement in which two different label colours are used. In this arrangement a single test line may be present which includes a combination of RBD and

SP. By using a multi-spectral sensor reader quantitative data and the ratio between the two antigens can be derived from the mixed colour characteristics.

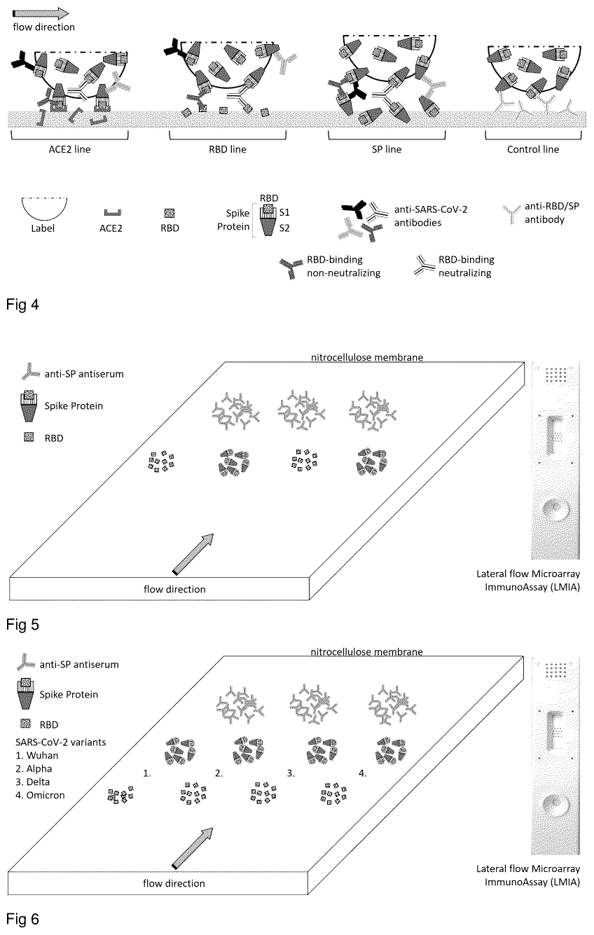

FIG. 4 shows an arrangement of three test lines in which the same label is used on the host receptor protein, the antigen fragment and the full length antigen.

FIG. 5 shows an arrangement in top view using an array of test spots rather than test lines. Here again the RBD label, the SP label, or a combination of RBD and SP labels may be used. In the test region duplo spots have been printed of both RBD and SP. Distal to these spots three control spots are present. On the right side an example is shown of such a lateral flow microarray immunoassay (LMIA).

FIG. 6 shows a lateral flow microarray test with three rows of spots; the lower row with SARS-COV-2 RBD antigen fragments from the original Wuhan virus and the Alpha, Delta, and Omicron variants, from left to right, the middle row with the SP full length antigens of these virus variants, and the upper row with three control spots. Here again the RBD label, the SP label, or a combination of RBD and SP labels may be used. On the right side an example is shown of such a lateral flow microarray immunoassay (LMIA).

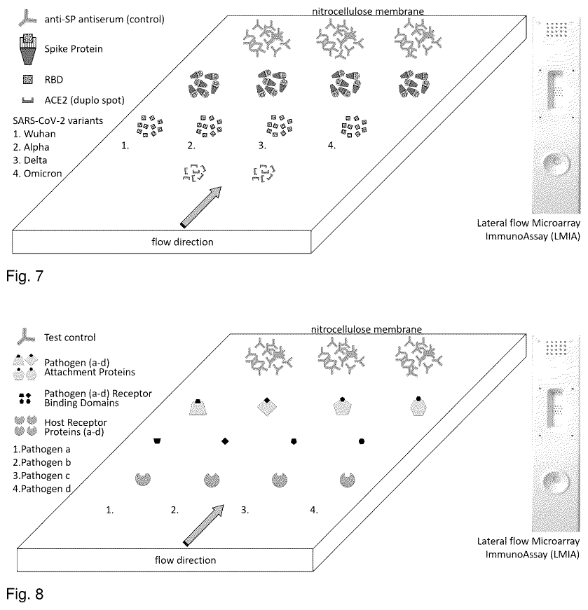

FIG. 7 shows a variation on the test of FIG. 6, In addition to the spots shown on the device of FIG. 5, this test also includes duplicate spots of immobilised human ACE2 receptor, to which the SARS-COV-2 SP binds in vivo; followed by RBD antigen fragment test spots, SP full length antigen test spots, and control test spots. Not shown is the conjugate pad, which is preloaded with labelled full length SP; adding the test sample to the conjugate pad allows both neutralising and non-neutralising RBD-binding antibodies to bind to the unlabelled full length SP. As the test sample flows across the test, it encounters the ACE2 receptor spots. Full length SP which is not bound by a neutralising antibody (including full length SP which is bound by a non-neutralising antibody) will bind to the ACE2 receptor, and so generate a detectable signal. The test sample also contacts the RBD spots, SP spots, and control spots as described elsewhere herein. The relative intensities of the receptor test spots and the antigen test spots can be determined, and potentially used to quantitate the titre of neutralising antibodies vs other antibodies in a sample. Labelled full length SP may be from one of the variants as present on the test; in this particular case Wuhan, Alpha, Delta, Omicron SP, which will lead to different results and conclusions.

FIG. 8 illustrates a similar arrangement, but having multiple different receptor test spots and multiple different pathogen test spots. Since different pathogens may interact with different host receptor proteins this arrangement allows simultaneous detection of and testing for neutralising antibodies against multiple different pathogens (for example, SARS, SARS-COV-2, MERS, and influenza).

### EXAMPLE

FIG. 1, lower panel, is useful to refer to here. From the origin of the test membrane (i.e. the side where the sample will be applied) the first line or spot includes immobilised antigenic proteins consisting of the Receptor Binding Domain (RBD) from the SARS-CoV-2 Spike Protein. The second line is the full Spike Protein and the third line is a test control, for example anti-RBD antibodies. This design can be adapted to any pathogen of which the entry protein and its sub-region that interacts with the host receptor is known. Such an entry protein is often the target of vaccine development. The test discriminates between antibodies binding the interactive sub-region from antibodies that bind other parts of the entry protein.

In the lateral flow test coloured (visible) or fluorescent nanoparticles will be used as test labels (other techniques such as magnetic and electrochemical technologies are possible). To these particles RBD or SP will be bound by physical adsorption or covalent interaction (RBD-, SP-conjugates). In the figure several options for binding the RBD- and SP-conjugates are illustrated. Both conjugates can bind to the two test lines, although the RBD-conjugate can only bind if anti-RBD antibodies are present. The SP-conjugates can be sandwiched to the membrane bound SP by all anti-SP antibodies (including anti-RBD antibodies) and to the membrane bound RBD by anti-RBD antibodies.

Both conjugates can bind to the control line where a generic target such as anti-RBD antibodies have been immobilised.

If we combine both conjugates in the test (FIG. 2), the anti-RBD antibodies in the human serum will be divided over the conjugates; part of these antibodies will bind to the RBD-conjugate and the remainder to the SP-conjugate. All other anti-SP antibodies can bind to the SP-conjugate. Since RBD is approximately 4 to 5 times smaller than SP there will be more RBD molecules on the RBD-conjugate than SP molecules on the SP-conjugate. Consequently, more anti-RBD antibodies may be attached to the RBD-conjugate. Therefore, for binding on the RBD line the RBD-conjugate may have a competitive advantage over the SP-conjugate. The level of this “advantage” can be further manipulated by changing the ratio between the two conjugates, for example, by addition of a larger volume of the RBD-conjugate as compared to the SP-conjugate.

If we would only use the RBD-conjugate, only the anti-RBD antibodies can bind. In this case the SP line will be left for binding complexes of anti-RBD antibodies and RBD-conjugate that were not bound at the RBD line.

If we would only use the SP-conjugate all anti-RBD and other anti-SP antibodies will bind. If the number of anti-RBD antibodies is high and/or if the combined affinity of the anti-RBD antibodies is strong there will be increased binding to the RBD line and less binding to the SP line. Other numbers and/or combined affinities will lead to other intensity ratios between the two test lines.

A combination of both conjugates will favour the anti-RBD antibodies due to the increased binding capacity of the RBD-conjugate. In this setup also anti-RBD antibodies bound to the SP-conjugate may bind to the RBD-line. All other anti-SP antibodies bound to the SP-conjugate can be bound on the SP line. The differences between these lines (kinetic and intensity) may correlate to neutralisation titers. To better correlate data to neutralisation titers other ratios in volume between the two conjugates may be considered.

The use of only the RBD-conjugate is not appropriate if it is necessary to have an SP line in addition to the RBD line. The presence of the SP line may help in elucidating the ratio between anti-RBD versus anti-‘SPminusRBD’ antibodies (numbers and combined affinities). If there are a lot of antibodies that recognise SP but not RBD the overwhelming number of anti-‘SPminusRBD’ antibodies may (sterically) hinder the binding of anti-RBD antibodies to the RBD region on SP (a kind of competition). This ‘competition’ may negatively influence the neutralisation titer.

The use of only the SP-conjugate is also possible, but lacks the advantage of the combination and, therefore, may not result in good correlation between test data and neutralisation titer. However, this may also be just the other way around; it may enlarge the ratio between anti-RBD and anti-‘SPminusRBD’ antibodies (the ‘competition’) which may lead to an even better correlation to neutralisation titers.

Inclusion of the receptor test line or spot, having human ACE2 receptor, provides improved discrimination between neutralising and non-neutralising antibodies. If neutralising antibodies are bound to the free labelled antigen (full length or fragment), then these antigens will be unable to bind to the immobilised ACE2 receptor, and no or a reduced signal will be formed at this line or spot. If other antibodies are bound to the antigen, it will still be available to bind to ACE2 receptor, so generating a signal, and also depleting the sample of non-neutralising antibodies as well as labelled antigen. This may improve sensitivity from the subsequent antigen test spots or lines, and allows for quantification of relative signal intensities, as well as measurement of kinetic properties as the signal develops over time. A preferred embodiment includes each of ACE2, RBD, and SP test spots or lines for a SARS-COV-2 test.

1. A method for detecting the presence in a test sample of a neutralising antibody to a neutralisation epitope of an antigenic protein, the method comprising:
applying a test sample to a sample receiving region of a lateral flow test device, the device comprising a solid support structure including the sample receiving region, a conjugate pad, an epitope test region and a receptor test region; wherein the conjugate pad comprises mobilisable neutralisation epitope conjugated to a detectable label, the epitope test region comprises immobilised neutralisation epitope which is not conjugated to a detectable label, and the receptor test region comprises immobilised receptor to the antigenic protein; and wherein the solid support structure is configured to permit liquid to flow from the sample receiving region via the conjugate pad to the test regions;
allowing the test sample to migrate to the conjugate pad and contact the neutralisation epitope to form labelled antibody-epitope complex if antibody is present;
allowing the test sample including labelled antibody-epitope complex or labelled epitope to migrate to the epitope and receptor test regions and contact the unlabelled antigen epitope or receptor respectively to form a complex, the label thereby providing a detectable signal; and
detecting a signal at the epitope test region, wherein presence of a signal is indicative of the presence of a neutralising antibody to said neutralisation epitope of an antigenic protein.

2. The method of claim 1 further comprising the step of detecting a signal at the receptor test region; and optionally comparing said signal to the signal detected at the epitope test region.

3. The method of claim 1 wherein the test sample comprises a body fluid (for example, blood, serum, saliva, mucus); or is a liquid sample derived from a swab or other test (for example, cellular and non-cellular material suspended in a liquid); or is a liquid sample derived from a solid sample (for example, a solution of the solid sample or some component thereof).

4. The method of claim 1 wherein the step of detecting the signal comprises quantitatively detecting the signal.

5. The method of claim 4 wherein the step of detecting the signal comprises quantitatively detecting development of the signal over time.

6. The method of claim 1 further comprising determining the kinetics of antibody binding based on the detected signal.

7. The method of claim 6 further comprising correlating the determined kinetics of antibody binding with the neutralising titre of the test sample.

8. The method of claim 1 wherein the mobilisable neutralisation epitope is comprised within a full length antigenic protein.

9. The method of claim 1 wherein the immobilised neutralisation epitope consists of a fragment of the full length antigenic protein, said fragment preferably being one which is known to be recognised by neutralising antibodies.

10. The method of claim 1 wherein the epitope test region further comprises immobilised full length antigenic protein not conjugated to a detectable label.

11. The method of claim 10 wherein detecting the signal comprises detecting signals from the antibody-labelled epitope-unlabelled epitope complex and from an antibody-labelled epitope-unlabelled full length protein complex, and the method further comprises comparing said detected signals.

12. The method of claim 9 wherein the epitope test region comprises both the fragment and the full length antigenic protein in a single test region, and each is labelled with a different detectable label.

13. The method of claim 9 wherein the epitope test region comprises first and second separate test regions, each of which includes either the fragment or the full length antigenic protein.

14. The method of claim 12 wherein the method further comprises comparing the binding kinetics of both the fragment and the full length antigenic protein.

15. The method of claim 1 wherein the neutralisation epitope is the SARS-COV-2 receptor binding domain (RBD) peptide.

16. The method of claim 1 wherein the detectable label is selected from the group consisting of a dye particle, a fluorescent label, a carbon particle, a latex particle, a gold particle, and a magnetic particle.

17. The method of claim 1 wherein multiple sequential epitope test regions are present.

18. The method of claim 1 wherein multiple parallel or otherwise non-sequential epitope test regions are present.

19. The method of claim 1 wherein the neutralisation epitope is a host-interacting domain of a pathogen protein, preferably wherein the pathogen protein is a host-interacting protein or a toxin; or is a viral protein, bacterial protein, phage protein, fungal protein; or is a parasite protein or an allergen.

20. The method of claim 19 wherein the receptor is a host receptor of the pathogen protein.

21. The method of claim 1 wherein the solid support further comprises a control region.

22. The method of claim 1 wherein the test region comprises immobilised neutralisation epitopes from a plurality of variants of a pathogen strain.

23. The method of claim 22 wherein the pathogen strain is selected from SARS-COV-2, SARS-COV, MERS, and influenza.

24. The method of claim 1 wherein the epitope test region comprises immobilised neutralisation epitopes from a plurality of different pathogens.

25. The method of claim 1, wherein the antigenic protein is a pathogen protein, and when used:
a) for measuring severity of a pathogenic infection in a patient;
b) for determining whether a subject has been exposed to a pathogenic infection;
c) for differential identification of immune responses to infections in a patient; wherein the test region comprises immobilised neutralisation epitopes from a plurality of different pathogens;
d) for determining efficacy of a vaccine, wherein the sample is obtained from a subject that has been administered a vaccine against the pathogen; and detection of a signal at the test region is indicative of an efficacious vaccination in a subject.

26. A lateral flow test device comprising a solid support structure including a sample receiving region, a conjugate pad, an epitope test region, and a receptor test region; wherein the conjugate pad comprises mobilisable neutralisation epitope conjugated to a detectable label, the epitope test region comprises immobilised neutralisation epitope which is not conjugated to a detectable label, and the receptor test region comprises immobilised receptor to the antigenic protein; and wherein the solid support structure is configured to permit liquid to flow from the sample receiving region via the conjugate pad to the test regions.

27. The lateral flow test device of claim 26 wherein the neutralisation epitope consists of a fragment of a full length protein, said fragment preferably being one which is known to be recognised by neutralising antibodies; and the epitope test region further comprises immobilised full length protein not conjugated to a detectable label.

28. The lateral flow test device of claim 26, further comprising, or provided in combination with, a reader device which is capable of detecting the detectable label.
